(19) United States
(12) Patent Application Publication
(10) Pub. No.: US 2024/0285851 A1
(43) Pub. Date: Aug. 29, 2024

(54) SKIN PATCH DRUG INFUSION DEVICE

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/571,721

(22) PCT Filed: Jul. 15, 2021
(86) PCT No.: PCT/CN2021/106462

§ 371 Date: Dec. 19, 2023

(30) Foreign Application Priority Data
Jul. 2, 2021 (WO) PCT/CN2021/104147

(51) Int. Cl. A61M 5/142

(52) U.S. Cl. CPC A61M 5/14248; A61M 2202/0007

(57) ABSTRACT

A skin patch drug infusion device includes: a control mechanism module and an infusion mechanism module. The infusion mechanism module includes a reservoir, a piston and a screw. A driving unit includes one rotating shaft and at least one driving member, and the driving member includes one driving end, the driving member can rotate around the rotating shaft to advance or reset the driving end. At least one driving wheel is provided with wheel teeth, and the advancing driving end can push the wheel teeth to rotate the driving wheel, thereby driving the screw forward. A linear actuator and a reset unit are respectively connected to the driving member, the linear actuator and the reset unit are respectively apply driving power to the driving member to advance and reset the driving end.

### TECHNICAL FIELD

The present invention mainly relates to the field of medical instruments, in particular to a skin patch drug infusion device.

### BACKGROUND

The pancreas in a normal person can automatically monitor the amount of glucose in the blood and automatically secrete the required dosage of insulin/glucagon. However, for diabetic patients, the function of the pancreas is abnormal, and the pancreas cannot normally secrete required dosage of insulin. Therefore, diabetes is a metabolic disease caused by abnormal pancreatic function and also a lifelong disease. At present, medical technology cannot cure diabetes, but can only control the onset and development of diabetes and its complications by stabilizing blood glucose.

Patients with diabetes need to check their blood glucose before injecting insulin into the body. At present, most of the detection methods can continuously detect blood glucose, and send the blood glucose data to the remote device in real time for the user to view. This detection method is called Continuous Glucose Monitoring (CGM), which requires the detection device to be attached to the surface of the patients' skin, and the sensor carried by the device is inserted into the subcutaneous tissue fluid for testing. According to the blood glucose (BG) level, the infusion device, as a closed-loop or semi-closed-loop artificial pancreas, injects the currently required insulin dose.

Currently, the infusion accuracy of the existing infusion device is relatively low, and the highest infusion accuracy is limited to the minimal infusion increment (unit infusion amount), which is determined by the advancing increment of the driving wheel. The user or the closed-loop system cannot flexibly choose the infusion mode to meet the body's actual requirements for drugs.

Therefore, the prior art urgently needs a drug infusion device with high infusion accuracy.

### BRIEF SUMMARY OF THE INVENTION

The invention discloses a skin patch drug infusion device, provided with at least two blocking walls, can precisely control the rotation amplitude of the driving member and improve the infusion accuracy of the infusion device. The user or the closed-loop system can flexibly select different infusion modes to precisely control the body fluid level to meet the needs of the body.

The invention discloses a skin patch drug infusion device, which comprises: a control mechanism module provided with multiple first engaging portions and a first electrical connection exposed on the surface of the control mechanism module; and an infusion mechanism module including a case, with a second electrical connection exposed on the surface of the case and second engaging portions that engaged with the first engaging portions, when the control mechanism module and the infusion mechanism module are assembled with each other, the first engaging portions and the second engaging portions are engaged, the first electrical connection and the corresponding second electrical connection electrically connected to each other, thereby electrically connecting the control mechanism module and the infusion mechanism module; the infusion mechanism module further includes a reservoir, a piston and a screw, the piston connected with the screw and arranged in the reservoir; a driving unit including one rotating shaft and at least one driving member, and the driving member includes one driving end, the driving member can rotate around the rotating shaft to advance or reset the driving end; at least one driving wheel provided with wheel teeth, and the advancing driving end can push the wheel teeth to rotate the driving wheel, thereby driving the screw forward; a linear actuator and a reset unit respectively connected to the driving member, the linear actuator and the reset unit respectively apply driving power to the driving member to advance and reset the driving end; and at least two blocking walls, arranged at one side of the driving unit to limit the advancing position of the driving unit.

According to an aspect of the present invention, the blocking wall is an elastic conductive member.

According to an aspect of the present invention, the elastic conductive member is one or a combination of conductive spring, conductive leaf spring, conductive rubber, or conductive silica gel.

According to an aspect of the present invention, the linear actuator includes an electrically driven linear actuator, an electrically heated linear actuator or an elastic member.

According to one aspect of the present invention, the reset unit includes an electrically driven linear actuator or an electrically heated linear actuator.

According to one aspect of the present invention, at least a blocking wall is arranged on the other side of the driving unit.

According to an aspect of the present invention, it further includes a control unit, jointly control the advancing end of the driving member with the blocking wall on the other side of the driving unit.

According to one aspect of the present invention, it further includes buzzer, non-closed arranged in the control mechanism module.

According to one aspect of the present invention, at least a sound-permeable outlet is provided in the housing of the control mechanism module.

According to one aspect of the present invention, the control mechanism module includes an upper housing and a lower housing, the sound-permeable outlet is arranged on the lower housing.

According to one aspect of the present invention, a waterproof sound-permeable membrane is disposed between the sound-permeable outlet and the buzzer.

According to one aspect of the present invention, the first engaging portions and the second engaging portions include one or more of hooks, blocks, holes, or slots that engaged with each other.

According to one aspect of the present invention, the first engaging portions and the second engaging portions are multiple first electrical contacts and multiple second electrical contacts.

According to one aspect of the present invention, one of the first electrical contacts or the second electrical contacts is a rigid metal pin or an elastic conductive member.

According to one aspect of the present invention, the type of the elastic conductive member includes conductive spring, conductive leaf spring, conductive rubber, or conductive silica gel.

According to one aspect of the present invention, one of the first electrical contacts is a rigid metal pin while one of the second electrical contacts is a conductive spring, a groove, within which a sealing element is provided, is disposed around the area where multiple second electrical contacts are disposed.

According to one aspect of the present invention, a plurality of connection ends is provided in the infusion mechanism module, and a plurality of compressed conductive springs are respectively electrically connected to the corresponding connection ends.

According to one aspect of the present invention, a flexible circuit board on which the connection ends are disposed is further provided in the infusion mechanism module.

According to one aspect of the present invention, the case includes upper case and lower case.

According to one aspect of the present invention, the lower case further includes an outward extending portion, and a block is provided on the outside of the outward extending portion.

According to one aspect of the present invention, a pressing portion is provided on the outside end of the outward extending portion.

Compared with the prior art, the technical solution of the present invention has the following advantages:

In the skin patch drug infusion device disclosed by the present invention, provided with at least two blocking walls in the infusion mechanism module, can precisely control the rotation amplitude of the driving member and improve the infusion accuracy of the infusion device. The user or the closed-loop system can flexibly select different infusion modes to precisely control the body fluid level to meet the needs of the body.

Furthermore, the reset unit includes an electrically driven linear actuator, an electrically heated linear actuator, or an elastic member. The magnitude of the driving power output by the linear actuator can be controlled by the current, therefore the power output is more stable, thus making the amplitude or rate of movement of the driving member more stable and controllable. In addition, when the reset unit is an elastic member, the driving member can be automatically reset without consuming electric energy, thereby reducing the power consumption and cost of the infusion device.

Furthermore, a control mechanism module provided with multiple first engaging portions and first electrical contacts exposed on the surface of the control mechanism module; the infusion mechanism module is provided with a plurality of second electrical contacts exposed on the surface of the case. The contact area of the electrical contact is small, which facilitates the mechanism module design and helps reducing the volume of the control mechanism module. Secondly, when the first engaging portions and the second engaging portions are engaged, the first electrical contact and the corresponding second electrical contact press against each other, thereby electrically connecting the control mechanism module and the infusion mechanism module. The control mechanism module and the infusion mechanism module are electrically connected to each other through the two kinds of electrical contacts pressing against each other, which facilitates the optimization of the circuit design and helps improving the reliability of the electrical connection.

Furthermore, the type of the elastic conductive member includes conductive spring, conductive leaf spring, conductive rubber, or conductive silica gel. An elastic conductive member, compared with a fixed contact, can further increase the reliability of the electrical connection.

Furthermore, the alarm is non-closed arranged in the control mechanism module, reducing the energy consumption of the alarm, optimizing the power consumption configuration of the infusion device and saving production costs.

Furthermore, a waterproof sound-permeable membrane is disposed between the sound-permeable outlet and the buzzer, which has high level waterproof and dustproof capacity, the protect grade can reach to IP68.

Furthermore, a flexible circuit board on which the connection ends are disposed is further provided in the infusion mechanism module. The shape of the flexible circuit board can be flexibly designed according to the internal mechanism module features in the infusion mechanism module, which helps optimizing the internal design of the infusion mechanism module.

Furthermore, the lower case further includes an outward extending portion, and a block is provided on the outside of the outward extending portion. The block can prevent the control mechanism module from detaching from the infusion mechanism module.

### BRIEF DESCRIPTION OF THE DRAWINGS

FIG. 1a is a schematic view of the control mechanism module from the front side according to an embodiment of the present invention;

FIG. 1b is a schematic view of the control mechanism module from the bottom side according to an embodiment of the present invention;

FIG. 1c is schematic view of the control mechanism module without upper housing from the front side according to an embodiment of the present invention;

FIG. 2a is a schematic view of the infusion mechanism module according to an embodiment of the present invention;

FIG. 2b is a side view of the assembly of the control mechanism module and the infusion mechanism module according to an embodiment of the present invention;

FIG. 2c is a schematic top view of the lower case of the infusion mechanism module according to an embodiment of the present invention;

FIG. 2d is a schematic top view of the lower case of the infusion mechanism module according to another embodiment of the present invention;

FIG. 3a and FIG. 3b are respectively schematic views of the internal mechanism module of the infusion mechanism module according to an embodiment of the present invention.

FIG. 4 is a schematic diagram of main components in the infusion mechanism module according to an embodiment of the present invention;

FIG. 5a-FIG. 5c are schematic diagrams of the driving member at the initial position, half-way position and full-way position according to an embodiment of the present invention.

### DETAILED DESCRIPTION

As mentioned above, the infusion accuracy of the existing infusion device is relatively low, and the highest infusion accuracy is limited to the minimal infusion increment (unit infusion amount) which is determined by the advancing increment of the driving wheel. The user or the closed-loop system cannot flexibly choose the infusion mode to meet the body's actual requirements for drugs.

In order to solve this problem, the present invention provides a drug infusion device. The infusion device is provided with at least two blocking walls, can precisely control the rotation amplitude of the driving member and improve the infusion accuracy of the infusion device. The user or the closed-loop system can flexibly select different infusion modes to precisely control the body fluid level to meet the needs of the body.

Various exemplary embodiments of the present invention will now be described in detail with reference to the drawings. The relative arrangement of the components and the steps, numerical expressions and numerical values set forth in the embodiments are not to be construed as limiting the scope of the invention.

In addition, it should be understood that, for ease of description, the dimensions of the various components shown in the figures are not necessarily drawn in the actual scale relationship, for example, the thickness, width, length or distance of certain units may be exaggerated relative to other mechanism modules.

The following description of the exemplary embodiments is merely illustrative, and is not intended to be in any way limiting the invention and its application or use. The techniques, methods and devices that are known to those of ordinary skill in the art may not be discussed in detail, but such techniques, methods and devices should be considered as part of the specification.

It should be noted that similar reference numerals and letters indicate similar items in the following figures. Therefore, once an item is defined or illustrated in a drawing, it will not be discussed further in following description of the drawings.

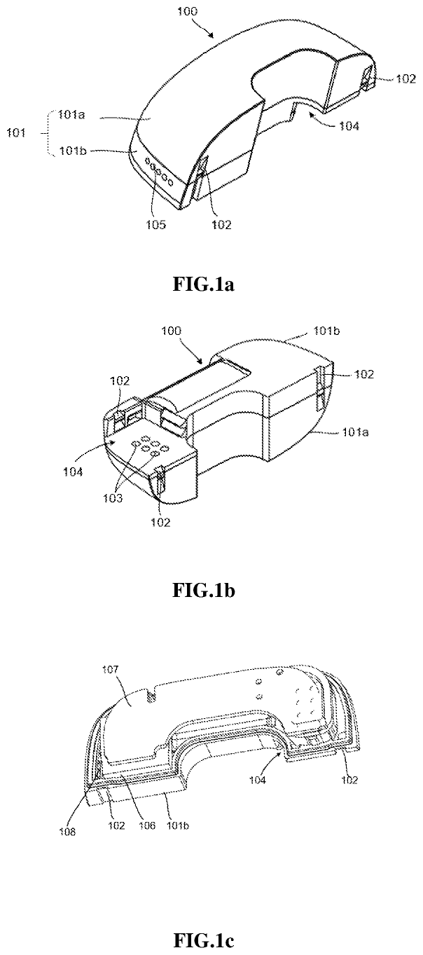

FIG. 1a is a schematic view of the control mechanism module from the front side according to an embodiment of the present invention; FIG. 1b is a schematic view of the control mechanism module from the bottom side according to an embodiment of the present invention; FIG. 1s is schematic view of the control mechanism module without upper housing 101a from the front side according to an embodiment of the present invention.

The skin patch drug infusion device of the embodiment of the present invention includes two main parts: a control mechanism module 100 and an infusion mechanism module 110, which will be described separately in detail below. And in another embodiment of the present invention, the skin patch drug infusion device can be provided with more than two parts, which is not specifically limited herein.

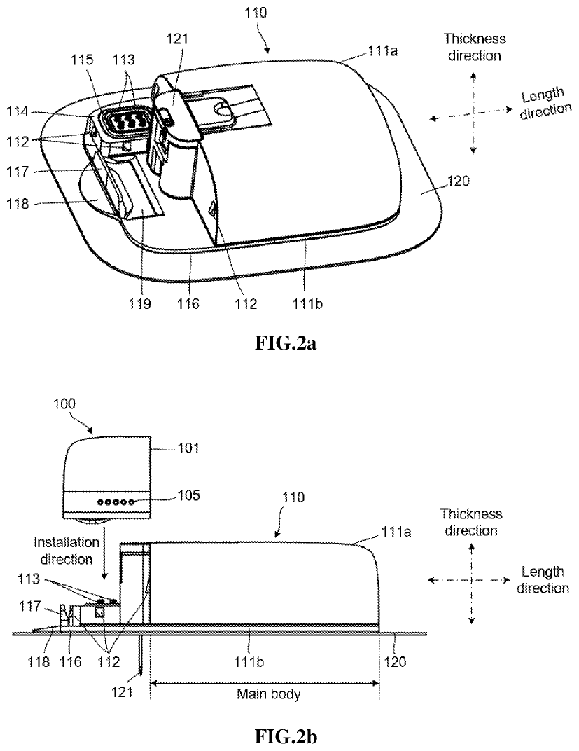

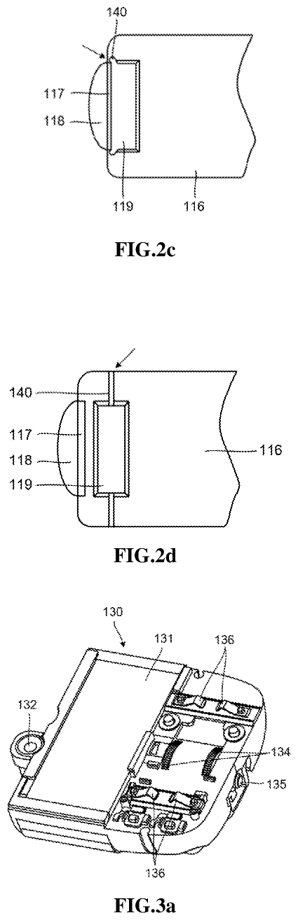

The skin patch drug infusion device refers to a tubing-free infusion device that is entirely pasted on the user's skin surface by the one piece of medical tape 120 (as shown in FIG. 2a and FIG. 2b). And the infusion device is provided with an infusion needle unit 121 (as shown in FIG. 2a and FIG. 2b), integrated on the infusion device, instead of a long tube, therefore, drug can be directly infused from the drug reservoir 131 (as shown in FIG. 3a and FIG. 3b) to the subcutaneous tissue through the infusion needle unit 121.

The skin patch drug infusion device of the embodiment of the present invention includes a control mechanism module 100 which receives signals or information from a remote device or a body fluid parameter detection device (such as CGM), and controls the infusion device to infuse drug(s) accordingly.

Inside the housing 101 of the control mechanism module 100 are disposed program modules, circuit board(s) 107 and related electronic units for receiving signals or issuing control instructions, as well as other mechanical units or structures necessary for realizing the infusion function, which is not limited herein. The housing 101 includes an upper housing 101a and a lower housing 101b. In another embodiment of the present invention, a power supply 133 can be also provided in the control mechanism module. Preferably, in the embodiment of the present invention, the power supply 133 is provided in the infusion mechanism module 110, which will be described below.

The control mechanism module 100 further includes a first electrical connection 103 exposed on the surface of it. The first electrical connection 103 is used as circuit connection terminal for electrically connecting the internal circuits provided in the control mechanism module 100 and the infusion mechanism module 110, respectively. The embodiment of the present invention does not specifically limit the positions of first electrical connection 103.

Preferably, in the embodiment of the present invention, the first electrical connection 103 is multiple first electrical contacts 103. Compared with the plug connector used as connection terminal in the prior arts, the contact area of the electrical contact is much smaller, which provides more flexibility to the mechanism module design, and can effectively reduce the volume of the control mechanism module. At the same time, these smaller electrical contacts can be directly electrically connected to the internal circuit or electrical components, or can be directly soldered on the circuit board, which helps to optimize the design of the internal circuit and effectively reduce the complexity of the circuit, thereby, saving costs and reducing the volume of the infusion device. Furthermore, the electrical contacts are exposed on the surface of the control mechanism module 100 to facilitate electrical connection with connection ends on other mechanism modules.

The type of the first electrical contact 103 includes rigid metal pins or elastic conductive members. Preferably, in the embodiment of the present invention, the first electrical contact 103 is a rigid metal pin. One end of the first electrical contact 103 is electrically connected to the connection end provided inside the control mechanism module 100 while the other end is exposed on the surface of the lower housing 101b. And the rest part of the first electrical contact 103 is tightly embedded in the housing 101, thus keeping the inside of the control mechanism module 100 isolated from the outside.

Here, the type of the elastic conductive member includes conductive spring, conductive silica gel, conductive rubber, or conductive leaf spring. Obviously, one end of the elastic conductive member is used to electrically connect with the internal connection end in the control mechanism module 100 while the other end is used to electrically connect with other connection ends. As in an embodiment of the present invention, the first electrical contact 103 is a conductive spring. When the electrical contacts are in contact with each other, the elasticity of the conductive spring can enhance the reliability of the electrical connection. Similar to the rigid metal pin, one end of the conductive spring is exposed on the surface of the lower housing 101b, while the rest part of the conductive spring is tightly embedded in the housing 101 and electrically connected with internal circuits or electrical components. Obviously, the connection end disposed inside the control mechanism module 100 can be a conductive lead, a specific part of a circuit, or an electrical element.

It should be noted that the “tightly embedded” in the embodiment of the present invention means that there is no gap between the electrical contact and the housing 101, keeping the control mechanism module 100 tightly sealed. The following “tightly embedded” has the same meaning as here.

In another embodiment of the present invention, the first electrical contact 103 is a conductive spring, but it is not tightly embedded in the housing 101. Instead, a sealing element is provided in a groove, both of which are disposed around the area where the first electrical contacts 103 are located, thus, sealing the electrical contact area and the control mechanism module 100.

In the embodiment of the present invention, the control mechanism module 100 is further provided with first engaging portions 102 which is used to fasten with the second engaging portion 112 disposed on the infusion mechanism module 110 to assemble the control mechanism module 100 and the infusion mechanism module 110, thereby enabling the electrical connection between the first electrical contacts 103 and the second electrical contacts 113 (as shown in FIG. 2a and FIG. 2b), which will be described in detail below.

The first engaging portion 102 and the second engaging portion 112 include one or more of hooks, blocks, holes, and slots that can be engaged with each other. The positions of the hooks, blocks, holes, and slots can be flexibly adjusted according to the shape and mechanism module features of the control mechanism module 100 and the infusion mechanism module 110, such as disposed in the interior or on the surface of the corresponding mechanism module, which is not specifically limited herein.

In the embodiment of the present invention, the control mechanism module 100 is further provided with a concave 104 that fits the convex portion 114 disposed at the bottom of the case of the infusion mechanism module 110, which will be described in detail below. Preferably, the first electrical contacts 103 are provided in the concave 104, as shown in FIG. 1b.

In the embodiment of the present invention, an alarm is non-closed provided in the control mechanism module 100, at least a non-closed area is also provided on the housing 101 of the control mechanism module 100. When the infusion process starts or ends, the infusion device malfunctions, the drug is exhausted, the control mechanism module 100 issues an error command or receives an error message, etc., the alarm is used to issue alarm signals, such as light, sound or vibration, notifying the user to adjust or replace the device in time. The alarm can be one or more of lighting lights, audio alarms, and vibration alarms, which is not specific limited here.

In the embodiment of the present invention, the non-closed area provided on the housing 101 of the control structure 100 is one or more openings, which can be a circle, a square, a triangle, a polygon, an irregular shape or any arbitrary shape, and arranged in a single row, multiple rows or any other random arrangement. The location of the non-enclosed area on the housing 101 of the control mechanism module 100 is not limited. Specifically, it may only be provided on the upper housing 101a or the lower housing 101b, or provided on the upper housing 101a and the lower housing 101b at the same time. The location of the non-closed area on the upper housing 101a or the lower housing 101b is also not limited. Specifically, it can be provided on the top, front, left or right side of the upper housing 101a or the lower housing 101b. It can also be provided on only one side, or provided on multiple sides at the same time. The number, shape, and arrangement of the non-closed area on each side are not limited, it can be the same or different. That is, the location, shape, size, number, and arrangement of the non-closed area are not limited here, as long as the alarm signal can be sent out from it. Preferably, in the embodiment of the present invention, the non-enclosed area is an opening provided along the edge of the alarm, that is, the location of the non-enclosed area is adapted to the position of the alarm, which makes it easier for the alarm signal to be sent out and raise the user's attention, reducing the energy consumption of the alarm, optimizing the power consumption configuration of the infusion device and saving production costs.

As the alarm is non-closed provided in the control mechanism module 100, compared with the traditional technical solution in which the alarm is entirely enclosed in the control mechanism module 100, when the alarm is a audio alarm, a less loud sound signal emitted from the alarm would be enough to raise the user's attention, which reduces the energy consumption of the audio alarm; When the alarm is a lighting alarm, it will be easier for the light to emit from the non-enclosed area and to raise the attention of the user, which reduces the energy consumption of the alarm. Especially, when the housing 101 of the control mechanism module 100 only can be made of a material with poor light transmission, more energy consumption of the alarm can be reduced as the light will be much easier to emit from the non-enclosed area. When the alarm is a vibration alarm, the weight of the infusion device is reduced due to the existence of the non-enclosed area, and the alarm requires only a small amount of power consumption to generate vibration and be noticed by the user. Based on the above mentioned reasons, when the alarm is a combination of audio alarm, lighting alarm, and vibration alarm, or an alarm that has multiple alarm signals such as lighting, sound, or vibration, as the alarm is non-closed provided in the control mechanism module 100, it will be easier for the alarm signals to emit from the non-enclosed area and to raise the attention of the user, reducing the energy consumption of the alarm, optimizing the power consumption configuration of the infusion device and saving production costs.

Specifically, in the embodiment of the present invention, the alarm is an audio alarm. Preferably, in the embodiment of the present invention, the audio alarm is a buzzer 106, amounted on the circuit board 107 of the control mechanism module 100. When the infusion process starts or ends, the infusion device malfunctions, the drug is exhausted, the control mechanism module 100 issues an error command or receives an error message, etc., the buzzer 106 is used to issue alarm signals, such as sound or vibration, notifying the user to adjust or replace the device in time. More specifically, in the embodiment of the present invention, the buzzer 106 is a piezoelectric buzzer.

Preferably, in the embodiment of the present invention, the non-closed area provided on the lower housing 101b of the control mechanism module 100 is a sound-permeable outlet 105 to allow the sound alarm signal from the buzzer 106 to be sent out. As mentioned above, the shape, size, number, arrangement on the housing 101 of the control mechanism module 100 of the sound-permeable outlet 105 are not limited, and will not be described here, as long as the sound alarm signal can be emitted. Preferably, in the embodiment of the present invention, the sound-permeable outlet 105 are a row of dense but separated small holes arranged on the side surface of the lower housing 101b. The diameter of the small holes is very small, smaller than the diameter of general water drops, which can prevent water drops from entering the control structure 101. The position of the sound-permeable outlet 105 is adapted to the position of the buzzer 106, and the overall length span is slightly larger than the edge length of the buzzer 106, so that the sound alarm signal of the buzzer 106 can be emitted more easily, reducing the energy consumption of the alarm, optimizing the power consumption configuration of the infusion device and saving production costs.

In order to achieve a good sealing effect and ensure the normal operation of the buzzer, a waterproof sound-permeable membrane 108 is disposed between the sound-permeable outlet 105 and the buzzer 106. Therefore, the waterproof sound-permeable membrane 108 needs to have a certain porosity to ensure the sound transmission but prevent water molecules penetration. Preferably, in the embodiment of the present invention, the waterproof sound-permeable membrane 108 is composed of a lot of small holes with a diameter of 0.1-1 microns, and the waterproof and dust-proof grade can reach to IP68.

Compared with the traditional technical solution in which the buzzer is entirely enclosed in the control mechanism module 100, because of the sound-permeable outlet 105 and the waterproof sound-permeable membrane 108, a less loud sound signal emitted from the buzzer would be enough to raise the user's attention, which reduces the energy consumption of the buzzer, thereby optimizing the power consumption configuration of the infusion device and saving production costs.

FIG. 2a is a schematic view of the infusion mechanism module 110 according to the embodiment of the present invention. FIG. 2b is a side view of the assembly of the control mechanism module 100 and the infusion mechanism module 110 according to the embodiment of the present invention. FIG. 2c is a schematic top view of the lower case of the infusion mechanism module according to an embodiment of the present invention. FIG. 2d is a schematic top view of the lower case of the infusion mechanism module according to another embodiment of the present invention.

The skin patch drug infusion device further includes an infusion mechanism module 110 with a case. A mechanical unit, an electric control unit, and other auxiliary units for completing drug infusion process are provided inside the case, which will be described in detail below. The case of the infusion mechanism module 110 may include multiple parts. As in the embodiment of the present invention, the case of the infusion device includes an upper case 111a and a lower case 111b.

As mentioned above, in the embodiment of the present invention, the infusion mechanism module 110 is provided with the second engaging portions 112 which is used to engaged and fasten with the corresponding first engaging portions 102. Therefore, the positions where the first engaging portion 102 and the second engaging portion 112 are provided correspond to each other.

In the embodiment of the present invention, the infusion mechanism module 110 is provided with a second electrical connection 113, the second electrical connection 113 is electrically connected to the first second electrical connection 103, thereby electrically connecting the control mechanism module 100 and the infusion mechanism module 100. Preferably, the second electrical connection 113 is multiple second electrical contacts 113. The technical advantages of the electrical contacts are applicable to both the first electrical contacts 103 on the control mechanism module 100 and the second electrical contacts 113 on the infusion mechanism module 110, which will not be described respectively in detail here. The second electrical contacts 113 are used to press against the corresponding first electrical contacts 103 to create electrical connection between the control mechanism module 100 and the infusion mechanism module 110. The mutual pressing between these two corresponding electrical contacts disposed on different mechanism modules can improve the reliability of the electrical connection. Similar to first electrical contacts 103, the type of one of the second electrical contact 113 also includes a rigid metal pin and an elastic conductive member. Preferably, in the embodiment of the present invention, the second electrical contact 113 is a conductive spring. Similarly, the conductive spring can improve the electrical connection performance. A groove is also arranged around the area where the second electrical contact 113 is disposed, and a sealing member 115 is arranged in the groove. Similarly, the elasticity of the conductive spring can further improve the electrical connection performance.

Preferably, in the embodiment of the present invention, the two ends of the conductive spring have different diameters. And the diameter of the end exposed to the outside of the infusion mechanism module 110 is shorter than that of the end inside the infusion mechanism module 110. In this way, the conductive spring can be held in the case because of the longer diameter, thus, when the control mechanism module 100 is not installed on the infusion mechanism module 110, the longer diameter of the inner end can prevent the conductive spring from detaching from the infusion mechanism module 110.

The embodiment of the present invention does not limit the position where second electrical contacts 113 are arranged, as long as it can be electrically connected to the corresponding first electrical contacts 103. Preferably, in the embodiment of the present invention, the upper case 111a of the infusion mechanism module 110 includes a convex portion 114 where the second electrical contacts 113 are disposed, as shown in FIG. 2a. The shape of the convex portion 114 corresponds to that of the concave 104 disposed on the control mechanism module 100, allowing the two portions to tightly fit each other and press the first electrical contacts 103 and the corresponding second electrical contacts 113 against each other to realize electrical connection.

In other embodiments of the present invention, the convex portion 114 may be provided on the lower case 111b, or when the infusion mechanism module 110 includes a integral case, the convex portion 114 is a part of the integral case, which is not specifically limited herein.

The method of assembling the control mechanism module 100 and the infusion mechanism module 110 to each other includes pressing the control mechanism module 100 on the infusion mechanism module 110 along with the thickness direction of the infusion mechanism module 110, thereby engaging the first engaging portion 102 and the second engaging portion 112. Or pressing the control mechanism module 100 on the infusion mechanism module 110 along with the length direction of the infusion mechanism module 110. Or alternatively, the control mechanism module 100 can be pressed along with any angle between the thickness direction and the length direction of the infusion mechanism module 110, making the first engaging portion 102 and the second engaging portion 112 engaged with each other. Preferably, in the implementation of the present invention, the method of which the control mechanism module 100 and the infusion mechanism module 110 are assembled with each other is to press the control mechanism module 100 on the infusion mechanism module 110 along with the thickness direction of the infusion mechanism module 110, making the first engaging portion 102 and the second engaging portion 112 engaged with each other, as shown the installation direction in FIG. 2b.

In the embodiment of the present invention, the lower case 111b of the infusion mechanism module 110 further includes an outward extending portion 116, and a block 117 is provided on the outside of the outward extending portion 116, as shown in FIG. 2a. As mentioned above, the control mechanism module 100 is pressed to the engaging position along the thickness direction of the infusion mechanism module 110, thus the block 117 can prevent the control mechanism module 100 from detaching along the length direction of the infusion mechanism module 110, ensuring the normal operation of the infusion device. Obviously, in another embodiment of the present invention, if the control mechanism module 100 is pressed to the engaging position along with other directions, the control mechanism module 100 can also be prevented from detaching from the infusion mechanism module 110 by adjusting the position of the block 117.

It should be noted here that “outward” and “outside” are relative to the main body of the infusion mechanism module 110, and belong to a concept of relative position, whose position relationship is shown in FIG. 2a or FIG. 2b. The “outside” below has the same meaning as here.

In the embodiment of the present invention, the outer end of the outward extending portion 116 is also provided with a pressing portion 118 for releasing the blocking effect of the block 117. While the user is replacing the infusion mechanism module 110, a finger presses the pressing portion 118, releasing the control mechanism module 100 from the block 117. Then, the user can remove the control mechanism module 100 from the infusion mechanism module 110 with another two fingers.

Another embodiment of the present invention can also be provided with an unlocking hole 119 disposed in the inner side of the block 117. While the pressing portion 118 is being pressed, a finger can enter the unlocking hole 119, thereby pushing the control mechanism module 100 out to separate the control mechanism module 100 from the infusion mechanism module 110. In the embodiment of the present invention, the unlocking hole 119 is square. The square unlocking hole 119 can facilitate smooth entry of fingers. In other embodiments of the present invention, the unlocking hole 119 may also have other shapes, which is not specifically limited here.

The lower case 111b of the infusion mechanism module 110 is also provided with one or more crease grooves 140. Two crease grooves 140 are provided on both sides of the unlocking hole 119, as shown in FIG. 2c and FIG. 2d. After the crease groove 140 is provided, the thickness or width of the lower case 111b at the position of the crease groove 140 (as shown by the arrows in FIG. 2c and FIG. 2d) is reduced. When the user presses the pressing portion 118, the lower case 1l1b is easy to be broken at the position of the crease groove 140, and the blocking of the control mechanism module 100 by the block 117 is more smoothly released.

Preferably, in the embodiment of the present invention, two crease grooves 140 are provided at the two ends of the block 117 respectively, as shown in FIG. 2c. In another embodiment of the present invention, the crease groove 140 is provided on two corresponding lateral sides of the unlocking hole 119, as shown in FIG. 2d.

The infusion mechanism module 110 of the embodiment of the present invention is further provided with an infusion needle unit 121 for infusing the drug under the skin.

A medical tape 120 is also provided on the bottom of the lower case 111b for attaching the infusion device on the surface of the user's skin.

FIG. 3a and FIG. 3b are respectively two schematic views of the internal mechanism module 130 of the infusion mechanism module 110 of the embodiment of the present invention from two perspectives.

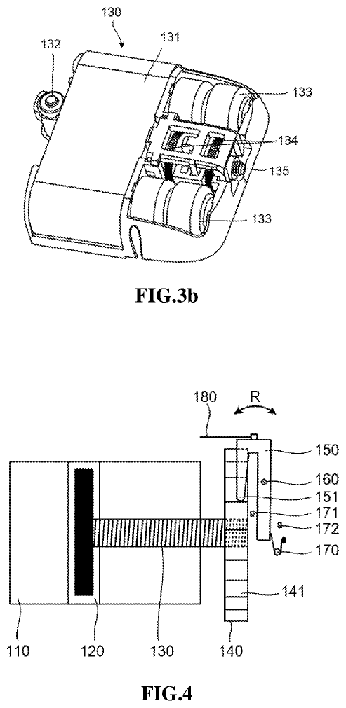

In the embodiment of the present invention, the internal mechanism module 130 includes mechanical units and electronic control units, used to realize the infusion function, such as a drug reservoir 131, a drug outlet 132, a power supply 133, a driving wheel 134, a screw 135, a circuit board 107 (as shown in FIG. 1c), a driving unit (not shown), etc. The movement of the driving unit drives the driving wheel 134 to rotate, thus making the screw 135 push the piston 1313 (as shown in FIG. 4) in the drug reservoir 131 to forward, realizing the drug infusion.

In the embodiment of the present invention, the power supply 133 is a conventional button battery. In other embodiments of the present invention, the power supply 133 may also be other types of batteries, as long as it can meet the requirements for supplying power to the infusion device. Preferably, in the embodiment of this present invention, the type of the power supply 133 is double-row battery pack, that is, two rows of button batteries are respectively arranged on both sides of the driving wheel 134, as shown in FIG. 3b. Conventionally, the discharge capacity of button batteries is low. The double-row button battery pack can reduce the discharge level of each battery, thereby extending the service life of the battery. Furthermore, the double-row design of the power supply 133 can make full use of the internal space and improve the integration of the internal mechanism module in the infusion device.

The infusion mechanism module 110 in the embodiment of the present invention is also provided with a circuit board or multiple three-dimensional circuits coated on the surface of a part of the mechanism module for supplying power to specific structural units. The circuit board is a hard/rigid circuit board or a flexible circuit board. Preferably, in the embodiment of the present invention, the circuit board is a flexible circuit board. The shape of the flexible circuit board is plastic, allowing it to be flexibly designed according to the internal space of the infusion mechanism module 110. At the same time, multiple connection ends can be provided on the flexible circuit board to be electrically connected to second electrical contacts 113, thereby connecting the circuits of the control mechanism module 100 and the infusion mechanism module 110, letting the infusion device to perform drug infusion function.

An elastic conductor 136 is also provided inside the infusion mechanism module 130, as shown in FIG. 3a. The elastic conductor 136 is electrically connected to the power supply 133 and the specific connection end on the circuit board (or three-dimensional circuit), thereby supplying power to specific structural units.

Similar to the elastic conductive member above mentioned, the type of the elastic conductor 136 includes a conductive spring, a conductive leaf spring, a conductive rubber, a conductive silica gel, etc., which are not specifically limited herein, as long as they can meet the requirements for electrically connecting the power supply 133 to specific connection ends on the circuit board (or three-dimensional circuit). Preferably, in the embodiment of the present invention, the elastic conductor 136 is the conductive leaf spring. Obviously, since the infusion mechanism module 110 has a double-row battery pack, the multiple conductive leaf springs are also designed as a double-row pack, as shown in FIG. 3a.

The elastic conductor 136 can realize direct electrical connection between the power supply 133 and the specific structural units, which helps to optimize the internal circuit design and reduce the complexity of the internal mechanism module.

FIG. 4 is a schematic diagram of main components in the infusion mechanism module 110 according to an embodiment of the present invention.

The internal component of the infusion mechanism module 110 mainly includes at least a reservoir 131, a piston 1313, a screw 135, a driving wheel 134, a driving unit (not shown), a reset unit 137 and a linear actuator 138. The driving unit includes at least one driving member 139 and at least one rotating shaft 1310. In the embodiment of this present invention, the driving member 139 is connected to the reset unit 137 and the linear actuator 138, respectively. It should be noted that the connection herein includes mechanical connection or electrical connection.

The reservoir 131 is used for storing liquid drug. Drugs include, but are not limited to, insulin, glucagon, antibiotics, nutrient solutions, analgesics, morphine, anticoagulants, gene therapy drugs, cardiovascular drugs or chemotherapy drugs, etc.

The piston 1313 is used to infuse liquid drug into the body.

The screw 135 is connected to the piston 1313 and the driving wheel 134, respectively. In the embodiment of the present invention, the driving wheel 134 advances the screw 135 forward by screwing, the screw 135 then forces the piston 1313, arranged in the reservoir 131, to move forward, so as to achieve the purpose of drug infusion. When the screw 135 is a flexible screw, the screw 135 may be designed to be curved. In one embodiment of the invention, the flexible screw is formed by a plurality of threaded sub-units movably connected one by one.

The peripheral surface of the driving wheel 134 is provided with wheel teeth 1341. The wheel teeth 1341 are gear teeth or ratchet teeth. Specifically, in the embodiment of the present invention, for improving driving efficiency, the wheel teeth 1341 are ratchet teeth which can be pushed more easily.

One driving end 1391 is provided on the driving member 139 to push the wheel teeth 1341, thereby rotating the driving wheel 134. The driving member 139 is movably connected to the rotating shaft 1310.

The linear actuator 138 and the reset unit 137 cooperate with each other to make the driving member 139 rotate reciprocally around the rotating shaft 1310, as shown in the R direction in FIG. 4, thereby making the driving end 1391 move in the advancing direction and reset direction. When the driving member 139 performs one reciprocating rotation, the driving end 1391 drives the driving wheel 134 forward one tooth, the driving wheel 134 drives the screw 135 forward one step, and the screw 135 engages the piston 1313 to infuse one unit of drug.

It should be noted here that the advancing direction of the driving end 1391 refers to the direction in which the wheel teeth 1341 moves, while the reset direction of the driving end 1391 is opposite to the advancing direction. During reset, the driving end 1391 only slides on the surface of the wheel teeth 1341 without pushing.

In some embodiments of the present invention, the reset unit 137 at least includes a spring, an elastic piece, an elastic plate, an elastic rod, rubber and other elastic members. It should be noted that the spring herein includes a compression spring, an extension spring, or a torsion spring, etc, so as to the meaning of the spring below. Specifically, in the embodiment of the present invention, the reset unit 137 is a torsion spring which is more conducive to the reset of the driving member 139. In some embodiments of the present invention, the reset unit 137 may also be an elastic conductive member, such as a metal spring, conductive rubber, or the like. In other embodiments of the present invention, the reset unit 137 includes an electrically driven linear actuator or an electrically heated linear actuator, such as a shape memory alloy. The type, material selection or the position of the reset unit 137 are not specifically limited herein, as long as it can satisfy the condition of making the driving member 139 rotate in the reset direction.

After being energized, the physical form of the material of the linear actuator, like shape memory alloy, changes, which makes shrinkage deformation of the shape memory alloy occur, thereby outputting the driving power. The higher the current is, the larger the shrinkage deformation of the shape memory alloy occurs, and the greater the driving power outputs. Obviously, when the current is constant, the amplitude of the driving power output by the shape memory alloy is also constant, therefore the shape memory alloy can output stable and controllable driving power for drug infusion.

The linear actuator 138 is an electrically driven linear actuator or an electrically heated linear actuator. By alternately turning on and off, the linear actuator 138 outputs power in pulses. Specifically, in the embodiment of the present invention, the linear actuator 138 is a shape memory alloy.

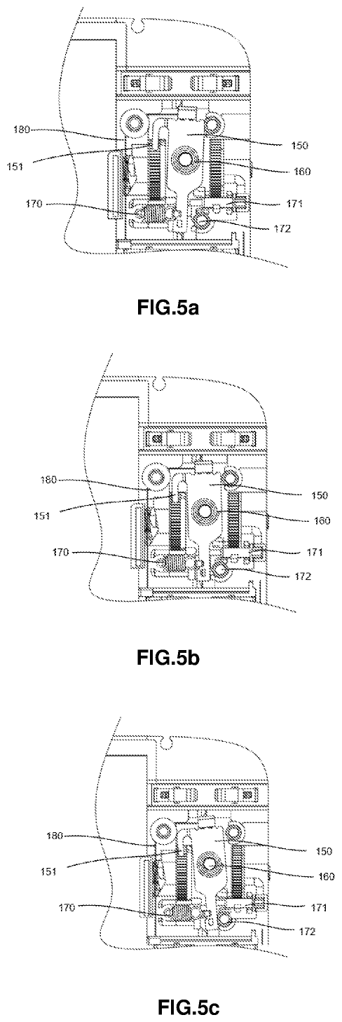

FIG. 5a-FIG. 5c are schematic diagrams of the driving member 150 at the initial position, half-way position and full-way position according to an embodiment of the present invention.

In the embodiment of the present invention, blocking walls 1311 and 1312 that can limit the advancing position of the driving member 139 are also provided in the infusion device, and blocking wall 1311 is a half-way blocking wall, blocking wall 1312 is a full-way blocking wall. When the driving member 139 is in the initial position, the driving member 139 is not contacted with the blocking wall. When the driving member 139 is in the half-way position, the driving member 139 is contacted with the blocking wall 1311. When the driving member 139 is in the full-way position, the driving member 139 is contacted with the blocking wall 1312. When the driving component 150 is contacted with the retaining wall 1311 or 1312, the electric potential of the contact point is changed, triggering an electrical signal, which will be transmitted to the control mechanism module 100 through a wire. The control mechanism module 100 determines whether the drive unit has reached the advancing end of the selected infusion mode. In the embodiment of the present invention, the driving wheel 134 drives the screw 135 forward one step, one unit of drug (unit infusion amount) infused by the infusion device, thus, in one reciprocating rotation, when the advancing end of the driving unit is the contact point between the driving unit and the half-way blocking wall 1311, ½ unit infusion amount can be infused, and the infusion devise can precisely control the rotation amplitude of the driving member 139, improve the infusion accuracy of the infusion device and meet the requirements of users or patients for different infusion accuracy.

Preferably, a first electrical signal is triggered when the linear actuator 138 pulls the driving member 139 forward and contact with the half-way blocking wall 1311, and the driving end 1391 pushes the driving wheel 134 forward ½ tooth, and the drive wheel 140 drives the screw 135 forward ½ step, the first electrical signal is transmitted to the control mechanism module 100 to determine whether the drive unit has reached the advancing end of the selected infusion mode. When the highest accuracy requirement is ½ unit infusion amount in the selected infusion mode, the control mechanism module 100 controls the linear actuator 138 to stop the power output, and the drive unit resets under the action of the reset unit; When the highest accuracy requirement is 1 unit infusion amount in the selected infusion mode, the control mechanism module 100 controls the linear driver 180 to continue to output power, and when the linear actuator 138 pulls the driving member 139 to continue to move forward, a second electrical signal is triggered when the linear actuator 138 pulls the driving member 139 forward, compress the half-way blocking wall 1311 and contact the full-way blocking wall 1312, the drive end 1391 pushes the drive wheel 140 forward another ½ tooth, the drive wheel 140 drives the screw 135 forward another ½ step, the control mechanism module 100 controls the linear drive 180 to stop the power output, and the drive unit resets under the action of the reset unit.

In the embodiment of the present invention, for example, when insulin is infused, if the unit infusion amount is 0.001 U, the infusion amount of 0.0005 U and 0.001 U can be completed in one reciprocating movement. Correspondingly, when the requirement of the selected infusion mode is 0.0015 U, the infusion can be completed by one full reciprocating movement and a half reciprocating rotation of the driving unit. Of course, when the requirement of selected infusion mode is 0.001 U*n+0.0005 U, the infusion can be completed by n times full reciprocating rotation and a half reciprocating rotation of the driving unit.

In another embodiment of the present invention, the number of blocking walls can also be three, namely, ⅓-way blocking wall, ⅔-way blocking wall, and full-way blocking wall, respectively. Correspondingly, ⅓ unit infusion amount can be infused in one infusion process, and the rotation amplitude of the driving member 150 can be further accurately controlled, the infusion accuracy can be improved, and the requirements of users or patients for different infusion accuracy can be met. Similarly, the number of blocking walls can also be 4 or more (n≥4). Correspondingly, the infusion accuracy can be increased to 1/n unit infusion amount. Theoretically, greater n, higher infusion accuracy, it can meet more requirements of different users or patients for different infusion accuracy. However, due to the requirement of miniaturization of the infusion device, too many blocking walls will increase the size and the design difficulty of the infusion device, therefore, 2-3 blocking walls will be better.

In the embodiment of the present invention, the blocking walls 1311 and 1312 are arranged on the same side of the driving unit, the reset unit 137 is an elastic member or an elastic conductive member, the other side of the driving unit is not provided with a blocking wall, and the advancing end of the driving member 139 on the other side is determined by the reset unit, or one or two blocking walls can be provided on the other side of the driving unit, and the advancing end of the driving member 139 on the other side is determined by the reset unit and the blocking wall.

In another embodiment of the present invention, the reset unit 137 is an electrically driven linear actuator or an electrically heated linear actuator, such as a shape memory alloy, and the blocking walls 1311 and 1312 are arranged on the same side of the driving unit, two blocking walls are correspondingly provided on the side of the driving unit to limit the advancing end of the driving member 139. Alternatively, it is also possible to provide only one blocking wall on the other side of the driving unit, and the advancing end of the driving member 139 on the other side is controlled by the control mechanism module 100 and the blocking wall, or no blocking wall provided on the other side of the driving unit, the advancing end of the driving member 139 on the other side is totally controlled by the control mechanism module 100.

Similarly, in the embodiment of the present invention, when the number of blocking walls on one side of the drive unit is more than two, the number of the blocking wall provided on the other side of the driving unit is not specifically limited here, as long as advancing end of the drive member 139 can be controlled.

In the embodiment of the present invention, the blocking wall is one or a combination of conductive spring, conductive leaf spring, conductive rubber, or conductive silica gel etc., preferably, in the embodiment of the present invention, the blocking wall is a pogo pin, and the blocking wall 1311 is arranged along the horizontal direction (paralleled to the screw), and the blocking wall 1312 is arranged in the vertical direction (perpendicular to the bottom of the infusion device), which can make full use of the internal space of the infusion device and optimize the design of the infusion device. The blocking wall 1311 is arranged closer to the driving unit than the blocking wall 1312, the driving unit can smoothly and sequentially contact with blocking wall 1311 and blocking wall 1311 to determine the advancing end of the driving member 139, and trigger the electrical signals to realize the high-accuracy delivery of the infusion device. In other embodiments of the present invention, the design, material and arrangement of each blocking wall are not specifically limited here, as long as it can contact with the driving unit to determine the advancing end of the driving member 139, and trigger the electrical signals to realize high-accuracy delivery of the infusion device.

In summary, the present invention discloses a skin patch drug infusion device, provided with at least two blocking walls, can precisely control the rotation amplitude of the driving member so that the minimum infusion amount is at least ½ unit infusion amount, which improve the infusion accuracy of the infusion device. The user or the closed-loop system can flexibly select different infusion modes to precisely control the body fluid level to meet the needs of the body, improving user experience.

While the invention has been described in detail with reference to the specific embodiments of the present invention, it should be understood that it will be appreciated by those skilled in the art that the above embodiments may be modified without departing from the scope and spirit of the invention. The scope of the invention is defined by the appended claims.

1. A skin patch drug infusion device, comprising:
a control mechanism module provided with multiple first engaging portions and a first electrical connection exposed on a surface of the control mechanism module; and
an infusion mechanism module including a case, with a second electrical connection exposed on a surface of the case and second engaging portions that engaged with the first engaging portions, wherein when the control mechanism module and the infusion mechanism module are assembled with each other, the first engaging portions and the second engaging portions are engaged, the first electrical connection and the second electrical connection are electrically connected to each other, thereby the control mechanism module and the infusion mechanism module are electrically connected to each other; the infusion mechanism module further includes:
a reservoir, a piston and a screw, the piston connected with the screw and arranged in the reservoir;
a driving unit including one rotating shaft and one driving member, wherein the driving member includes one driving end, and the driving member rotates around the rotating shaft to advance or reset the driving end;
one driving wheel provided with wheel teeth, the driving end pushes the wheel teeth to rotate the driving wheel, thereby driving the screw forward;
a linear actuator and a reset unit respectively connected to the driving member, wherein the linear actuator and the reset unit respectively apply driving power to the driving member to advance and reset the driving end; and
at least two first blocking walls, arranged at one side of the driving unit to limit an advancing position of the driving unit.

2. A skin patch drug infusion device of claim 1, wherein
each of the first blocking walls is an elastic conductive member.

3. A skin patch drug infusion device of claim 2, wherein
the elastic conductive member at least comprises a conductive spring, a conductive leaf spring, a conductive rubber, or a conductive silicone.

4. A skin patch drug infusion device of claim 1, wherein
the reset unit includes an electrically driven linear actuator, an electrically heated linear actuator or an elastic member.

5. A skin patch drug infusion device of claim 4, wherein
at least a second blocking wall is arranged on an other side of the driving unit.

6. A skin patch drug infusion device of claim 5,
further comprising a control unit, jointly control the driving end of the driving member with the second blocking wall on the other side of the driving unit.

7. A skin patch drug infusion device of claim 1,
further comprising a buzzer, non-closed arranged in the control mechanism module.

8. A skin patch drug infusion device of claim 7, wherein
a sound-permeable outlet is provided on a housing of the control mechanism module.

9. A skin patch drug infusion device of claim 8, wherein
the housing of the control mechanism module includes an upper housing and a lower housing, the sound-permeable outlet is arranged on the lower housing.

10. A skin patch drug infusion device of claim 9, wherein
a waterproof sound-permeable membrane is disposed between the sound-permeable outlet and the buzzer.

11. A skin patch drug infusion device of claim 1, wherein
each of the first engaging portions and the second engaging portions includes a hook, a block, a hole, or a slot, and each of the first engaging portions and the corresponding second engaging portion are engaged with each other.

12. A skin patch drug infusion device of claim 11, wherein
the first engaging portions and the second engaging portions are first electrical contacts and second electrical contacts.

13. A skin patch drug infusion device of claim 12, wherein
one of the first electrical contacts or the second electrical contacts is a rigid metal pin or an elastic conductive member.

14. A skin patch drug infusion device of claim 13, wherein
the elastic conductive member includes a conductive spring, a conductive leaf spring, a conductive rubber, or a conductive silicone.

15. A skin patch drug infusion device of claim 14, wherein
one of the first electrical contacts is a rigid metal pin while one of the second electrical contacts is a conductive spring, a groove, within which a sealing element is provided, is disposed around an area where the second electrical contacts are disposed.

16. A skin patch drug infusion device of claim 15, wherein
a plurality of connection ends are provided in the infusion mechanism module, and a plurality of compressed conductive springs are respectively electrically connected to the connection ends.

17. A skin patch drug infusion device of claim 16, wherein
a flexible circuit board on which the connection ends are disposed is further provided in the infusion mechanism module.

18. A skin patch drug infusion device of claim 1, wherein
the case includes an upper case and a lower case.

19. A skin patch drug infusion device of claim 18, wherein
the lower case further includes an outward extending portion, and a block is provided on an outside of the outward extending portion.

20. A skin patch drug infusion device of claim 19, wherein
a pressing portion is provided on the outside of the outward extending portion.
